(19) United States
(12) Patent Application Publication
(10) Pub. No.: US 2017/0260157 A1
(43) Pub. Date: Sep. 14, 2017

(54) PROCESS FOR PREPARATION AND PURIFICATION OF POMALIDOMIDE

(71) Applicant: Apicore US LLC, Somerset, NJ (US)

(72) Inventors: Ravishanker Kovi, Monroe, NJ (US); Veerabhadra Rao Bobbili, Andhra Pradesh (IN); Jayaraman Kannapan, Gujarat (IN)

(73) Assignee: Apicore US LLC, Somerset, NJ (US)

(21) Appl. No.: 15/452,444

(22) Filed: Mar. 7, 2017

(30) Foreign Application Priority Data
Mar. 14, 2016 (IN) 201621008822

(51) Int. Cl. C07D 401/04

(52) U.S. Cl. CPC C07D 401/04

(57) ABSTRACT

Processes are disclosed for making pomalidomide which involve reducing 2-(2,6-dioxopiperidin-3-yl)-4-nitroisoindoline-1,3-dione using a catalyst and at least one solvent. The process may include reacting 3-nitrophthalic anhydride with α-amino glutarimide hydrochloride to obtain the 2-(2,6-dioxopiperidin-3-yl)-4-nitroisoindoline-1,3-dione. The process may further include, prior to the reducing step, subjecting the 2-(2,6-dioxopiperidin-3-yl)-4-nitroisoindoline-1,3-dione to a purification process comprising heating a mixture of 2-(2,6-dioxopiperidin-3-yl)-4-nitroisoindoline-1,3-dione and 1,4-dioxane to obtain a solution, treating the obtained solution with carbon, removing the carbon from the solution to obtain a purified 2-(2,6-dioxopiperidin-3-yl)-4-nitroisoindoline-1,3-dione solution and using the purified 2-(2,6-dioxopiperidin-3-yl)-4-nitroisoindoline-1,3-dione solution for the reducing step. Processes disclosed achieve pomalidomide having a purity of greater than 99% as measured by HPLC with no individual impurity present in an amount greater than 0.1% and total impurities comprising not more than 0.5%.

### CROSS-REFERENCE TO RELATED APPLICATIONS

This non-provisional application claims the benefit of priority of Indian Provisional Patent Application No. 201621008822, filed on Mar. 14, 2016, the entirety of which is incorporated herein by reference.

### FIELD OF THE INVENTION

The present invention relates to processes for preparation of pomalidomide. In particular, the present invention relates to processes for purifying pomalidomide and pomalidomide having a purity of at least 95% prepared using novel techniques.

### BACKGROUND

Pomalidomide (POMALYST®) is an immunomodulatory antineoplastic agent. The chemical name is (RS)-4-amino-2-(2,6-dioxo-piperidin-3-yl)-isoindoline-1,3-dione and has the following chemical structure:

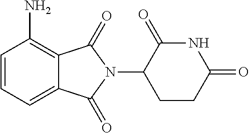 [SMILES: Nc1cccc2c1C(=O)N(C1CCC(=O)NC1=O)C2=O]

Pomalidomide inhibits LPS-induced monocyte TNFα, IL-1β, IL-12, IL-6, MIP-1, MCP-1, GM-CSF, G-CSF, and COX-2 production, and may be used in treating various disorders for example as disclosed in U.S. Pat. Nos. 5,635,517, 6,316,471 and 6,476,052. The compound is also known to co-stimulate the activation of T-cells. Pomalidomide has direct anti-myeloma tumoricidal activity, immunomodulatory activities and inhibits stromal cell support for multiple myeloma tumor cell growth.

Specifically, pomalidomide inhibits proliferation and induces apoptosis of hematopoietic tumor cells. Additionally, pomalidomide inhibits the proliferation of lenalidomide-resistant multiple myeloma cell lines and synergizes with dexamethasone in both lenalidomide-sensitive and lenalidomide-resistant cell lines to induce tumor cell apoptosis. Pomalidomide enhances T cell- and natural killer (NK) cell-mediated immunity, and inhibits production of pro-inflammatory cytokines (e.g., TNF-α and IL-6) by monocytes. Pomalidomide also inhibits angiogenesis by blocking the migration and adhesion of endothelial cells. Due to its diversified pharmacological properties, pomalidomide is useful in treating, preventing, and/or managing various diseases or disorders.

### SUMMARY OF THE INVENTION

Embodiments of the presently disclosed subject matter relate to methods of synthesis of pomalidomide which produce pomalidomide in high yield and purity. Embodiments disclosed herein also relate to processes for purification of pomalidomide which produce highly pure pomalidomide. In at least one embodiment a process is disclosed which produces pomalidomide having a purity greater than 95%. In at least one embodiment a process is disclosed which produces pomalidomide having a purity greater than 99% as measured by HPLC.

Processes disclosed herein are simple, economical and industrially viable and produce pure pomalidomide in high yields.

In accordance with an embodiment a process for making pomalidomide includes reducing 2-(2,6-dioxopiperidin-3-yl)-4-nitroisoindoline-1,3-dione using a catalyst and at least one solvent selected from 1,4-dioxane, water, acetic acid, tetramethylurea (TMU), N,N′-dimethyl ethylene urea (DMEU), N,N-dimethyl propylene urea (DMPU) or mixtures thereof to obtain 4-amino-2-(2,6-dioxopiperidin-3-yl) isoindoline-1,3-dione (pomalidomide), and optionally purifying the pomalidomide.

The process may include the step of reacting 3-nitrophthalic anhydride with α-amino glutarimide hydrochloride to obtain the 2-(2,6-dioxopiperidin-3-yl)-4-nitroisoindoline-1,3-dione. The reaction of 3-nitrophthalic anhydride with α-amino glutarimide hydrochloride may be carried out in the presence of sodium acetate and acetic acid.

In one or more embodiments, the process may include, prior to reducing 2-(2,6-dioxopiperidin-3-yl)-4-nitroisoindoline-1,3-dione, subjecting the 2-(2,6-dioxopiperidin-3-yl)-4-nitroisoindoline-1,3-dione to a purification process which includes heating a mixture of 2-(2,6-dioxopiperidin-3-yl)-4-nitroisoindoline-1,3-dione and 1,4-dioxane to obtain a solution, treating the obtained solution with carbon, removing the carbon from the solution to obtain a purified 2-(2,6-dioxopiperidin-3-yl)-4-nitroisoindoline-1,3-dione solution and using the purified 2-(2,6-dioxopiperidin-3-yl)-4-nitroisoindoline-1,3-dione solution for the reducing step.

In or more embodiments the catalyst may be palladium on carbon (Pd/C) or Raney nickel and the solvent selected from the group consisting of 1,4-dioxane, water, acetic acid and mixtures thereof.

For example, and not by way of limitation, in one embodiment when the catalyst is Pd/C the solvent is 1,4-dioxane. In another embodiment when the catalyst is Pd/C or Raney nickel the solvent is a mixture of 1,4-dioxane and water. In yet a further embodiment when the catalyst is Pd/C the solvent is a mixture of 1,4-dioxane, water and acetic acid. In still further embodiments when the catalyst is Pd/C the solvent is acetic acid.

The reaction time for reduction using a palladium catalyst may be in the range of 1 to 12 hr, while the reaction time for reduction using a Raney nickel catalyst may be in the range of 60 to 90 hr.

In accordance with some embodiments the process of making pomalidomide may include subjecting the pomalidomide to a purification process involving suspending pomalidomide in one or more of 1,4-dioxane, water, ethyl acetate or a mixture thereof, optionally heating the suspension, and recovering purified pomalidomide from the suspension. The process may include suspending the pomalidomide in a mixture of 1,4-dioxane and ethyl acetate, heating the suspension, filtering the suspension to obtain a wet pomalidomide material; combining the wet pomalidomide material with ethyl acetate under reflux conditions, stirring the pomalidomide material combined with the ethyl acetate, cooling the stirred material to room temperature, filtering and subsequently washing the cooled material to obtain purified pomalidomide.

In still further embodiments a process for making pomalidomide may include subjecting the pomalidomide to a purification process including the steps of a) suspending pomalidomide in TMU to obtain a suspension, b) heating the suspension to obtain a clear solution, c) treating the clear solution obtained in step b) with carbon, then removing the carbon prior to step d), d) adding an anti-solvent to the solution obtained in step c), and e) recovering purified pomalidomide. The ratio of pomalidomide to TMU be about 20 volumes of TMU to one gram of pomalidomide. The anti-solvent used in step d) may be water, methanol, acetone, cyclohexane, diisopropyl ether, ethyl acetate, toluene, methyl tertiary butyl ether, acetic acid or a mixture thereof.

In still yet a further embodiment, a process for making pomalidomide includes subjecting pomalidomide to a purification process including the steps of a) suspending pomalidomide in TMU, b) heating the suspension, c) subsequently cooling the suspension, d) stirring the cooled suspension, e) subjecting the suspension of step d) to filtration to obtain a solid product, f) leaching the solid product with water, and g) recovering purified pomalidomide. In this embodiment the ratio of pomalidomide to TMU is about 5 volumes of TMU to one gram of pomalidomide.

Processes disclosed herein yield pomalidomide having a purity of 95% or greater as measured by high pressure liquid chromatography. In some embodiments pomalidomide made according to the presently disclosed processes have a purity of greater than 98% as measured by high pressure liquid chromatography. In still further embodiments processes disclosed herein yield pomalidomide having a purity of greater than 99% as measured by HPLC with no individual impurity present in an amount greater than 0.1% and total impurities not more than 0.5%.

### BRIEF DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a graphical depiction of a high pressure liquid chromatography (HPLC) chromatogram of pomalidomide made according to a process in accordance with one or more embodiments of the present invention;

FIG. 2 is graphical depiction of an X-ray powder diffraction pattern of pomalidomide made according to a process in accordance with one or more embodiments of the present invention; and

FIG. 3 is a graphical depiction of a HPLC chromatogram of pomalidomide made according to a process in accordance with one or more embodiments of the present invention.

### DETAILED DESCRIPTION OF THE INVENTION

Embodiments of the present invention now will be described more fully hereinafter with reference to the accompanying examples and experiments, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All percentages and ratios used herein are by weight of the total composition and all measurements made are at 25° C. and normal pressure unless otherwise designated. All temperatures are in degrees Celsius unless specified otherwise.

There are two major problems with pomalidomide synthesis. One is to synthesize pomalidomide having high purity which is at least 95% and above, preferably at least 99% and above. Another problem is to synthesize pomalidomide with less than 1% of total impurities.

There are several processes disclosed in the art for preparation of pomalidomide and its purification. WO2014170909 discloses a process for the preparation of pomalidomide crystalline Form I, which includes suspending pomalidomide in dimethyl formamide, dimethyl acetamide or dimethyl sulfoxide; heating the suspension above 70° C.; adding anti-solvent to the obtained solution; and isolating pomalidomide crystalline Form I. WO2014170909 also discloses a process for purification of pomalidomide which includes suspending pomalidomide in an organic solvent; heating the suspension at reflux; treating the solution obtained with carbon; cooling the solution; optionally adding an alcoholic solvent, water or an acetic acid; and isolating the pomalidomide.

WO2015075694 discloses a process for preparing pomalidomide which involves reaction of nitro phthalic acid with 3-amino-piperidine-2,6-dione or its salt in the presence of a coupling agent and a first solvent to obtain 3-(3-nitrophthalimido)-piperidine-2,6-dione; and reduction of the 3-(3-nitrophthalimido)-piperidine-2,6-dione in the presence of a second solvent and a catalyst to obtain pomalidomide. WO2015075694 also discloses purification of pomalidomide by dissolving pomalidomide in an organic solvent selected from the group consisting of dimethyl sulfoxide, diethyl sulfoxide, di-n-propyl sulfoxide, di- or tetra-n-butyl sulfone sulfoxide, acetone, methyl isobutyl ketone, and mixtures thereof adding an anti-solvent selected from alcohol, ether, water, and mixtures thereof; and isolating the pomalidomide.

Chinese patent applications CN104910132, CN104557858, CN104402863, and CN104387366 also disclose processes for preparation of pomalidomide. Chinese patent applications CN104557857, CN103288797 and CN103275062 disclose methods of purification of pomalidomide.

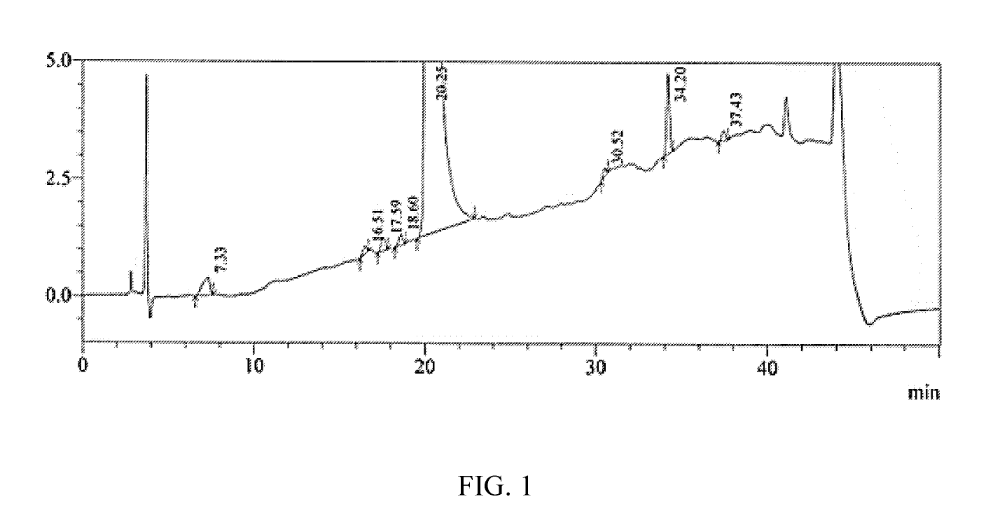

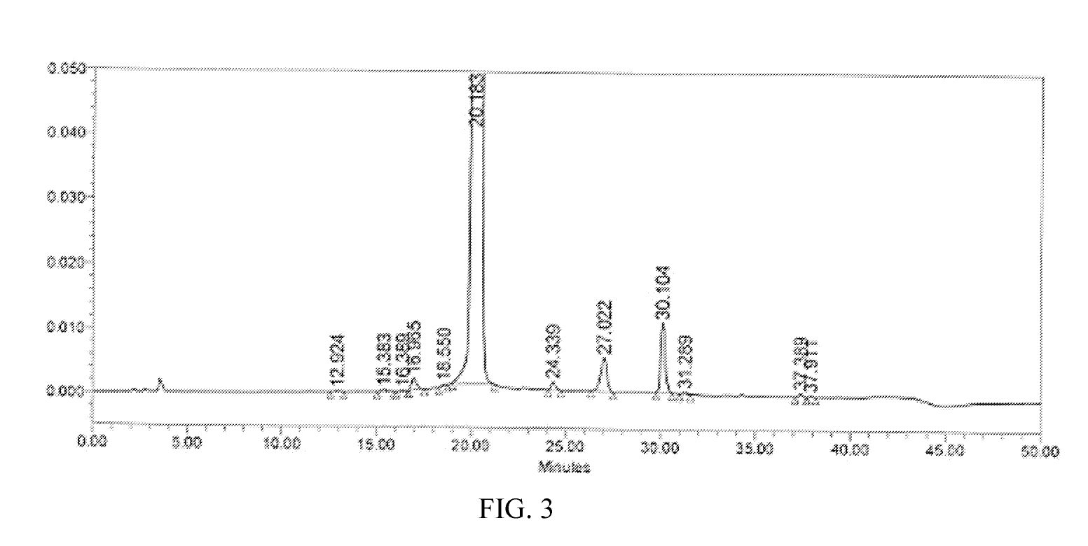

The present inventors found some prior art processes for making pomalidomide result in formation of unknown (unidentified) specified impurity along with the pomalidomide which eluted at RRT 1.5±0.1. This impurity was present at greater than 0.5% and at times even greater than 1%. Processes disclosed herein achieve pomalidomide having a purity of 99% and above, total impurities below 1% and the unknown specified impurity eluting at RRT 1.5±0.1 less than 0.5%, in some embodiments below 0.1% and in some embodiments less than 0.05%. For example, FIG. 1 reflects a batch made according to an embodiment herein wherein purity of pomalidomide was greater than 99% and the specified impurity was 0.01%. FIG. 3 reflects a batch of pomalidmide prepared according to the presently disclosed methods wherein purity of pomalidomide was greater than 99% and the specified impurity was about 0.43%.

According to certain embodiments a process for preparation of pomalidomide involves preparation of 2-(2,6-dioxopiperidin-3-yl)-4-nitroisoindoline-1,3-dione; and preparation of 4-amino-2-(2,6-dioxopiperidin-3-yl)isoindoline-1,3-dione (pomalidomide) by reduction of 2-(2,6-dioxopiperidin-3-yl)-4-nitroisoindoline-1,3-dione in presence of catalyst and one or more solvent selected from 1,4-dioxane, water, acetic acid, aprotic solvent selected from tetramethylurea (TMU), N,N′-dimethyl ethylene urea (DMEU), N,N-dimethyl propylene urea (DMPU) or mixtures thereof.

According to an aspect, a process of preparation of pomalidomide includes the steps of a) reacting 3-nitrophthalic anhydride with α-amino glutarimide hydrochloride to give 2-(2,6-dioxopiperidin-3-yl)-4-nitroisoindoline-1,3-dione; b) reducing the 2-(2,6-dioxopiperidin-3-yl)-4-nitroisoindoline-1,3-dione in the presence of a catalyst selected from palladium on carbon or Raney nickel and one or more solvents selected from 1,4-dioxane, water and acetic acid to give pomalidomide; and c) optionally purifying the pomalidomide.

In step a) the reaction of 3-nitrophthalic anhydride with α-amino glutarimide hydrochloride is carried out in the presence of sodium acetate and acetic acid to form 2-(2,6-dioxopiperidin-3-yl)-4-nitroisoindoline-1,3-dione. The reaction is performed at temperature greater than 50° C., preferably about 100-120° C. It takes about more than 8-10 hours, preferably around 15-25 hours. The compound obtained in step a) is further suspended in water once or twice, the slurry is filtered and dried to obtain pure 2-(2,6-dioxopiperidin-3-yl)-4-nitroisoindoline-1,3-dione. The compound, 2-(2,6-dioxopiperidin-3-yl)-4-nitroisoindoline-1,3-dione obtained according to the presently disclosed process has greater than 95% purity, preferably greater than 98% purity when tested by HPLC.

In step b) the reduction of 2-(2,6-dioxopiperidin-3-yl)-4-nitroisoindoline-1,3-dione is carried out using a suitable catalyst. Preferably, the catalyst is selected from palladium on carbon (Pd/C) or Raney nickel. When the catalyst is Raney nickel a suitable amount is from 5 to 25% by weight of 2-(2,6-dioxopiperidin-3-yl)-4-nitroisoindoline-1,3-dione. When a Pd/C catalyst is used a suitable amount is from 5-10% by weight 2-(2,6-dioxopiperidin-3-yl)-4-nitroisoindoline-1,3-dione.

The solvent used for the reduction reaction can include one or more solvents selected from 1,4-dioxane, acetic acid and water. For example, and not by way of limitation, the solvent used for reduction using a Pd/C catalyst may be 1,4-dioxane; the solvent used for reduction using a Pd/C or Raney-Ni catalyst may be 1,4-dioxane and water; the solvent used for reduction using a Pd/C catalyst may be 1,4-dioxane, water and acetic acid; the solvent used for reduction using a Pd/C catalyst may be acetic acid.

The reduction reaction temperature is room temperature. The term “room temperature” refers to temperature at about 20 to 35° C. Preferably the temperature is 20-30° C. The reduction reaction is carried out at hydrogen pressure from about 60-80 psi. The reduction reaction time can be in the range of 1.5 hours to 90 hours. The reaction time for reduction using a Pd/C catalyst is in the range of 1 to 12 hours. The reaction time for reduction using a Raney nickel catalyst is in the range of 60-90 hours.

In some embodiments, in step b) the compound, 2-(2,6-dioxopiperidin-3-yl)-4-nitroisoindoline-1,3-dione is first subjected to purification and then reduced. This purification is performed to remove the unwanted impurities formed during the reaction. The purification involves heating a mixture of 2-(2,6-dioxopiperidin-3-yl)-4-nitroisoindoline-1,3-dione and 1,4-dioxane to 80° C. to get a solution, treating the obtained solution with carbon, stirring, filtering and using the this solution for the reduction reaction.

In step c) pomalidomide is subjected to purification by suspending pomalidomide in one or more solvents selected from 1,4-dioxane, water and ethyl acetate, optionally heating, and isolating pure pomalidomide which is free from unwanted impurities.

For example, purification of pomalidomide may include suspending pomalidomide in 1,4-dioxane and ethyl acetate, heating the suspension to 80° C. and filtering the material; treating the wet material with ethyl acetate under reflux conditions, stirring the treated wet material, cooling it to room temperature, filtering and washing to get purified pomalidomide.

According to another embodiment, a process for preparation of pomalidomide includes the steps of a) reacting 3-nitrophthalic anhydride with α-amino glutarimide hydrochloride to give 2-(2,6-dioxopiperidin-3-yl)-4-nitroisoindoline-1,3-dione; b) reducing 2-(2,6-dioxopiperidin-3-yl)-4-nitroisoindoline-1,3-dione in the presence of a palladium catalyst and one or more solvents selected from 1,4-dioxane, water and acetic acid to give pomalidomide; and c) optionally purifying pomalidomide.

According to another embodiment, a process for preparation of pomalidomide includes the steps of a) reacting 3-nitro phthalic anhydride with α-amino glutarimide hydrochloride to give 2-(2,6-dioxopiperidin-3-yl)-4-nitroisoindoline-1,3-dione; reducing 2-(2,6-dioxopiperidin-3-yl)-4-nitroisoindoline-1,3-dione in the presence of a Raney nickel catalyst and 1,4-dioxane and water as solvent to give pomalidomide; and optionally purifying pomalidomide.

In one or more embodiments, a process for preparation of pomalidomide involve reaction of 3-nitrophthalic anhydride with α-amino glutarimide hydrochloride in the presence of sodium acetate and glacial acetic acid at reflux temperature, preferably at a temperature of 118° C. for more than 10 hours, preferably, 17 to 24 hours. The reaction mass is cooled to lower the temperature and the solvent is removed. The wet material is subjected to purification by slurrying once or twice in water and further drying. The compound, 2-(2,6-dioxopiperidin-3-yl)-4-nitroisoindoline-1,3-dione thus obtained is further purified by heating in an organic solvent such as 1,4-dioxane, treating with activated carbon, cooling the mixture to 25-30° C. and filtering the mixture to get the solution containing 2-(2,6-dioxopiperidin-3-yl)-4-nitroisoindoline-1,3-dione. Additionally one or more solvents such as 1,4-dioxane and/or water are added to this solution containing 2-(2,6-dioxopiperidin-3-yl)-4-nitroisoindoline-1,3-dione followed by addition of a Raney nickel catalyst. The reaction mixture is stirred at a temperature of 20-30° C. under 60-80 psi hydrogen pressure for 60-90 hours. The Raney nickel catalyst loading is 25% by weight of 2-(2,6-dioxopiperidin-3-yl)-4-nitroisoindoline-1,3-dione. In some embodiments the catalyst is added in portions of 5% by weight of reactant five times to avoid the formation of impurities and each time the reaction is continued for 12 hours. The reduction reaction using a Raney nickel catalyst can be completed in 60-80 hours. After the completion of the reaction pomalidomide is isolated by filtering the reaction mass and removing the solvent under vacuum. The solid thus obtained is heated in one or more solvents such as water, 1,4-dioxane and ethyl acetate, stirred and filtered to obtain the pure compound. Pomalidomide thus obtained is at least 95% pure as measured by HPLC, and in some embodiments at least 99% pure as measured by HPLC.

In an alternative embodiment, pomalidomide is prepared by reducing the compound 2-(2,6-dioxopiperidin-3-yl)-4-nitroisoindoline-1,3-dione using a palladium catalyst (5-10% by weight of 2-(2,6-dioxopiperidin-3-yl)-4-nitroisoindoline-1,3-dione) in the presence of one or more solvents such as 1,4-dioxane, water and/or acetic acid for 1-12 hours, preferably 1-6 hours under 60-80 psi hydrogen pressure at a temperature of 20-30° C.; and isolating pomalidomide. The process provides pomalidomide in high yield and high HPLC purity. Pomalidomide thus obtained is at least 95% pure as measured by HPLC, and in some embodiments at least 99% pure as measured by HPLC.

In another embodiment a process for preparation of pomalidomide includes reduction of the compound, 2-(2,6-dioxopiperidin-3-yl)-4-nitroisoindoline-1,3-dione using a catalyst selected from Pd/C and Raney nickel in the presence of one or more aprotic solvents selected from tetramethylurea (TMU), N,N′-dimethyl ethylene urea (DMEU), and N,N′-dimethyl propylene urea (DMPU) for 6-12 hours under 60-80 psi hydrogen pressure at temperature 20-30° C.; and isolating pomalidomide. Accordingly, preparation of pomalidomide in this embodiment involves the steps of charging 2-(2,6-dioxopiperidin-3-yl)-4-nitroisoindoline-1,3-dione in TMU and heating the mixture up to 80° C. to 90° C. to get a clear solution. This clear solution is treated with charcoal at 80° C. to 90° C. The reaction mass is then cooled to 25-35° C. and a catalyst such as Pd/C is added to it. The reaction mixture is stirred at 20° C. to 30° C. under 70 psi hydrogen pressure for about 6 hours. The reaction mass is filtered and washed with a solvent such as TMU. To the reaction mass water is added and the mass stirred at 25° C. to 30° C. for about 3 hours. The precipitated solid is filtered and washed with water. The wet cake of pomalidomide is further slurried with water, the slurry filtered and the compound dried.

The process of the present invention is represented in Scheme 1 below.

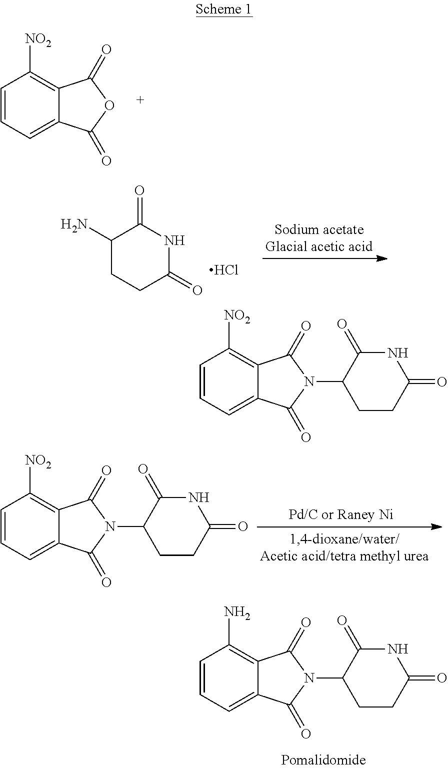

According to another embodiment a process for purification of pomalidomide includes the steps of a) suspending pomalidomide in tetra methyl urea to obtain a suspension; b) heating the suspension of step a) to get a clear solution; c) treating the solution of step b) with carbon; d) adding anti-solvent to the solution obtained in step c); and e) isolating purified pomalidomide.

In step a) pomalidomide is suspended in about 20 volumes of the solvent. Typically the ratio of pomalidomide to treating solvent is about 20 volumes of solvent per gram of pomalidomide. In step b) the suspension of pomalidomide in tetra methyl urea is heated at temperature 80-100° C. for 2-4 hours. The carbon treatment in step c) involves cooling the solution of step b) at about 50° C., adding activated charcoal, stirring and filtering. The anti-solvent used in step d) is one or more solvent selected from water, methanol, acetone, cyclohexane, diisopropyl ether, ethyl acetate, toluene, methyl tertiary butyl ether and acetic acid. The isolation of purified pomalidomide in step e) involves cooling the reaction mass to 30-40° C., stirring, filtering and drying the purified compound by conventional methods such as under vacuum or at reduced pressure. The compound thus obtained can be further treated or leached with preheated water at about 50° C. to remove tetra methyl urea completely.

In one embodiment the pomalidomide is purified by suspending pomalidomide in 20 volumes of tetra methyl urea, heating the suspension at 80-100° C. to get the clear solution, cooling the solution to about 50° C. and treating the solution with activated charcoal. The reaction mass is filtered and washed with tetra methyl urea at 50° C. The reaction mass is then heated at 80° C., the compound is precipitated using water. The reaction mass is stirred at 35° C. to 40° C. for about 4 hours, filtered and washed with water at 40° C. The wet cake is taken in preheated water at 50° C., stirred for 1 hour at 50° C., filtered, washed with water and dried. This water treatment can be repeated two or three times to remove tetra methyl urea completely.

Alternatively, crude pomalidomide may be purified by treating/leaching pomalidomide with less volume of tetra methyl urea followed by treating/leaching with water. For example, in one embodiment, pomalidomide is suspended in tetra methyl urea, preferably 5 volumes of the solvent, and the suspension heated up to 100° C. for 3 hours. The reaction mass is cooled, stirred and filtered. The mass is further treated/leached with water, filtered and dried to get pomalidomide having a purity of 95% or more, and preferably a purity of 99% or more.

Advantageously the purification processes disclosed herein provide pomalidomide with a purity of more than 99%, wherein residual solvents are within limits as per ICH guidelines. The present inventors have found that the use of tetra methyl urea for purifying pomalidomide effectively reduces residual 1,4-dioxane level in the final product. In one or more embodiments processes disclosed herein provide pomalidomide in which the residual 1,4-dioxane is well below the ICH limit of 380 ppm. Moreover, the processes disclosed herein provide pomalidomide having high purity of more than 99% by HPLC with all the individual impurity not more than 0.1% and the total impurity not more than 0.5%.

According to another embodiment, there is provided a process for the purification of pomalidomide, which includes heating the suspension of pomalidomide in tetra methyl urea at temperature of 80-100° C. and adding anti-solvent to get pure pomalidomide.

The purity of pomalidomide is determined by high performance liquid chromatography (HPLC) unless otherwise noted herein. Pomalidomide obtained according processes disclosed herein has a purity greater than 99%. The HPLC purity data for an exemplary embodiment is shown in FIG. 1 and Table 1, reflecting a purity of 99.88%. The impurity eluting at RRT 1.5±0.1 is 0.1%.

TABLE 1

| | Ret. | | | |
|---|---|---|---|---|
| Peak Name | Time | Area | Area % | RRT |
| | 7.33 | 10941 | 0.03 | 0.36 |
| | 16.51 | 2561 | 0.01 | 0.82 |
| | 17.59 | 4670 | 0.01 | 0.87 |
| | 18.60 | 3684 | 0.01 | 0.92 |
| Pomalidomide | 20.25 | 38816903 | 99.88 | 1.00 |
| | 30.52 | 2158 | 0.01 | 1.51 |
| | 34.20 | 19398 | 0.05 | 1.69 |
| | 37.43 | 3003 | 0.01 | 1.85 |
| Sum | | 38863318 | 100.00 | |

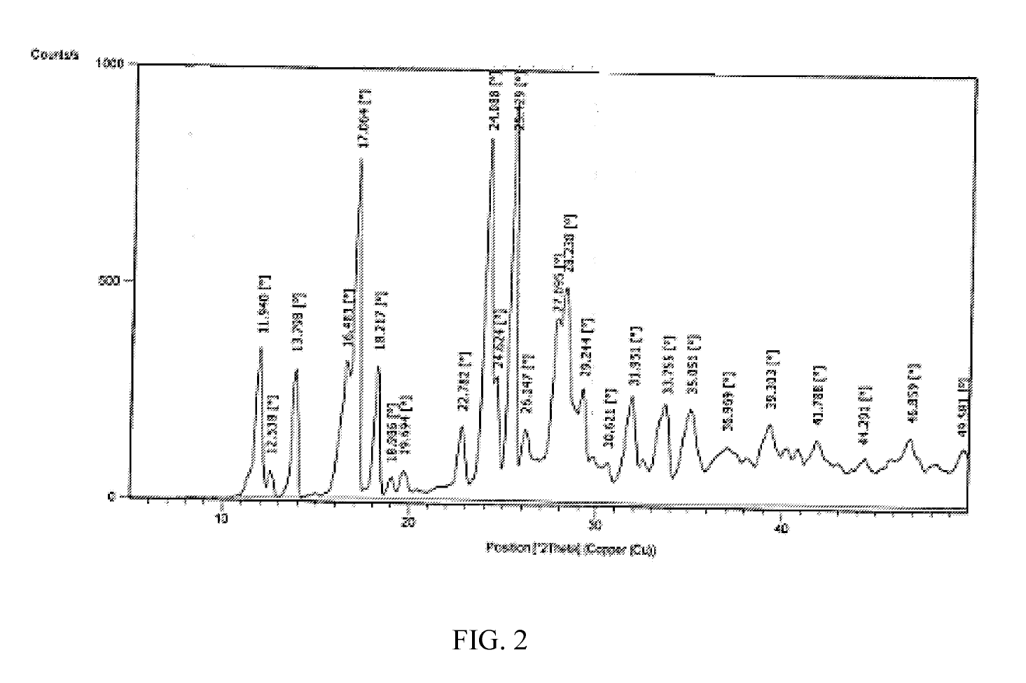

Pomalidomide prepared by process disclosed herein is a crystalline form having the x-ray powder diffraction pattern substantially as shown in FIG. 2 and is characterized by x-ray diffraction peaks at about 11.9, 12.5, 13.8, 17.0, 18.2, 22.7, 24.0, 24.6, 25.4, 28.2, 29.2±0.2 degrees. With reference to FIG. 2, in some embodiments a crystalline form of pomalidomide prepared according to the present invention has x-ray diffraction peaks at about 11.94, 12.53, 13.79, 16.48, 17.06, 18.21, 18.98, 19.69, 22.78, 24.08, 24.62, 25.42, 26.14, 27.69, 28.23, 29.24, 30.62, 31.95, 33.75, 35.05, 36.96, 39.30, 41.78, 44.29, 46.85, 49.58±0.2 20 degrees.

FIG. 3 is HPLC chromatogram of pomalidomide with a specified impurity at RRT 1.5±0.1 for an exemplary embodiment is as shown in FIG. 3 and Table 2. The purity is 99.06% and the impurity eluting at RRT 1.5±0.1 is less than 0.5%.

TABLE 2

| Peak Name | RT | Area | % Area | RRT |
|---|---|---|---|---|
| Peak 1 | 12.92 | 2290 | 0.01 | 0.64 |
| Peak 2 | 15.38 | 5943 | 0.01 | 0.76 |
| Peak 3 | 16.39 | 2453 | 0.01 | 0.81 |
| Peak 4 | 16.97 | 35198 | 0.09 | 0.84 |
| Peak 5 | 18.55 | 2630 | 0.01 | 0.92 |
| Pomalidomide | 20.18 | 39679885 | 99.06 | 1.00 |
| Peak 7 | 24.34 | 18890 | 0.05 | 1.21 |
| Peak 8 | 27.02 | 124571 | 0.31 | 1.34 |
| Peak 9 | 30.10 | 172452 | 0.43 | 1.49 |
| Peak 10 | 31.29 | 4759 | 0.01 | 1.55 |
| Peak 11 | 37.39 | 6995 | 0.02 | 1.85 |
| Peak 12 | 37.91 | 2231 | 0.01 | 1.88 |
| Sum | | 40058297 | 100.00 | |

The present invention is further illustrated by reference to the following examples which does not limit the scope of the invention in any way.

### Examples

### Example 1: Preparation of 2-(2,6-dioxopiperidin-3-yl)-4-nitroisoindoline-1,3-dione

A round bottom flask was charged with a solution of glacial acetic acid (75 ml) and α-amino glutarimide hydrochloride (8.5 g). Sodium acetate anhydrous (4.5 g) was added lot-wise to the solution at 25° C. to 30° C. followed by addition of 3-nitro phthalic anhydride (log) at the same temperature. The reaction mixture was stirred at 118° C. for 18 hr. After the completion of reaction, the reaction mass was cooled to 60° C. and the solvent was distilled off under vacuum to get the residue. To the residue obtained, water (100 ml) was added; the mixture was stirred for 1 hr at 25° C. to 30° C. and the mass filtered. The wet cake obtained was slurried with water (100 ml×2), filtered and dried in an air tray dryer until the water content was less than 0.5% to afford 2-(2,6-dioxopiperidin-3-yl)-4-nitroisoindoline-1,3-dione (14 g). Yield: 89.7%, Purity: 98%.

### Preparation of 4-amino-2-(2,6-dioxopiperidin-3-yl) isoindoline-1,3-dione (pomalidomide)

### Example 2: Preparation of 4-amino-2-(2,6-dioxopiperidin-3-yl) isoindoline-1,3-dione

A round bottomed flask was charged with 1,4-dioxane (600 ml) and 2-(2,6-dioxopiperidin-3-yl)-4-nitroisoindoline-1,3-dione (10 g). The mixture was stirred at 25° C. to 30° C. for 15 minutes, the temperature was raised up to 80° C. and the reaction mass stirred to obtain a clear solution. To the clear solution activated charcoal (5 g) was added at 80° C., the mixture stirred and the solution cooled to 25° C. to 30° C. The reaction mass was further stirred for 20 minutes at this temperature and filtered. To the obtained solution 1,4-dioxane (300 ml), water (180 ml) and Raney nickel (0.5 g) were added. The reaction mixture was stirred at 20° C. to 25° C. under 70 psi hydrogen pressure for 12 hrs. Raney nickel (0.5 g) was added to the reaction mixture at 20° C. to 25° C. and the reaction continued at the same temperature and pressure for 12 hrs. Another portion of Raney nickel (0.5 g) was added to the reaction mixture and the reaction continued at the same temperature and pressure for 12 hr. Another portion of Raney nickel (0.5 g) was added to the mixture and the reaction continued at the same temperature and pressure for 12 hr. Another portion of Raney nickel (0.5 g) was added to the reaction mixture and the reaction continued at the same temperature and pressure for 12 hr. The reaction mixture was then filtered through a celite bed and the solvent was distilled off from the reaction mixture under vacuum at 50° C. to obtain a solid. To the solid obtained 1,4-dioxane (50 ml) and ethyl acetate (5 ml) were added and the mixture was heated to 80° C. with stirring for 1 hr. The reaction mass was filtered at 80° C. and the obtained cake washed with 1,4-dioxane (10 ml) at 80° C. To the wet cake ethyl acetate (100 ml) was added and the mixture was refluxed with stirring for 1 hr. Then the reaction mass was cooled to 25° C. to 30° C., the solid filtered, washed with ethyl acetate (20 ml) and dried under vacuum to get 4-amino-2-(2,6-dioxopiperidin-3-yl) isoindoline-1,3-dione (7 g). Yield: 77.7% Purity: 99%.

### Example 3: Preparation of 4-amino-2-(2,6-dioxopiperidin-3-yl) isoindoline-1,3-dione

A round bottomed flask was charged with 1,4-dioxane (600 ml) and 2-(2,6-dioxopiperidin-3-yl)-4-nitroisoindoline-1,3-dione (10 g). The reaction mixture was stirred at 25° C. to 30° C. for 15 minutes, the reaction temperature was raised to 80° C. and the reaction mass stirred at this temperature to get a clear solution. To the clear solution obtained activated charcoal (5 g) was added at 80° C. The mixture was stirred and cooled to 25° C. to 30° C. The reaction mass was further stirred at the same temperature for 20 minutes and filtered. To the obtained solution 1,4-dioxane (300 ml), water (180 ml) and Raney nickel (1.0 g) were added. The reaction mixture was stirred at 20° C. to 25° C. under 70 psi hydrogen pressure for 12 hrs. Another portion of Raney nickel (1.5 g) was added to the reaction mixture and the reaction continued at the same temperature and pressure for 80 hr. The reaction mass was then filtered through a celite bed and the solvent was distilled off from the reaction mass under vacuum at 50° C. to get a solid. To the solid obtained 1,4-dioxane (50 ml) and ethyl acetate (5 ml) were added and the mixture was heated to 80° C. with stirring for 1 hr. The reaction mass was filtered at 80° C. and the cake washed with 1,4-dioxane (10 ml) at 80° C. To the wet cake ethyl acetate (100 ml) was added and the mixture was refluxed with stirring for 1 hr. The mixture was then cooled to 25° C. to 30° C., filtered to obtain the solid, which was washed with ethyl acetate (20 ml) and dried under vacuum to afford the titled compound (7 g). Yield: 77.7% Purity: 99%

### Example 4: Preparation of 4-amino-2-(2,6-dioxopiperidin-3-yl) isoindoline-1,3-dione

A round bottomed flask was charged with 2-(2,6-dioxopiperidin-3-yl)-4-nitroisoindoline-1,3-dione (10 g) and 1,4-dioxane (600 ml) was added to it. The mixture was first stirred at room temperature (25° C. to 30° C.) for 15 minutes, then the temperature was raised up to 80° C. and the mixture stirred to get a clear solution. To this solution activated charcoal (5 g) was added at 80° C. and cooled to 25° C. to 30° C. The reaction mass was stirred for 20 minutes and the reaction mass filtered. To the obtained solution 1,4-dioxane (300 ml) and water (180 ml) were added followed by addition of Raney Ni (4.5 g). The mixture was stirred at 20° C. to 25° C. under 70 psi hydrogen pressure for 12 hr. The reaction was monitored by TLC. After completion of the reaction, the reaction mass was filtered through a celite bed and the solvent was distilled off from the reaction mass under vacuum at 50° C. to obtain a residue. To this residue 1,4-dioxane (50 ml) and ethyl acetate (5 ml) were added and the mixture was stirred at 80° C. for 1 hr. The reaction mass was filtered at 80° C. and washed with 1,4-dioxane (10 ml) at 80° C. To the wet cake was charged ethyl acetate (100 ml) and the temperature raised to reflux. The reaction mass was refluxed with stirring for lhr and cooled to 25° C. to 30° C. The reaction mass was filtered and washed with ethyl acetate (20 ml) to afford the titled compound (7 g). Yield: 77%, Purity: 99.54%

### Example 5: Preparation of 4-amino-2-(2,6-dioxopiperidin-3-yl) isoindoline-1,3-dione

A round bottomed flask was charged with 1,4-dioxane (600 ml) and 2-(2,6-dioxopiperidin-3-yl)-4-nitroisoindoline-1,3-dione (10 g). The reaction mass was stirred at room temperature (25° C. to 30° C.) for 15 minutes and the temperature raised up to 80° C. and the reaction mass stirred at this temperature to get a clear solution. To the clear solution activated charcoal (5 g) was added at 80° C. and the mass was cooled to 25° C. to 30° C. The reaction mass was stirred at this temperature for 20 minutes and filtered. To the obtained solution palladium on carbon (Pd/C, 1.0 g, 10%) was added. The mixture was stirred at 20° C. to 30° C. under 70 psi hydrogen pressure for 12 hrs. The reaction mass was then filtered through a celite bed and the solvent distilled off from the reaction mass under vacuum at 50° C. to get the solid. To the solid obtained 1,4-dioxane (50 ml) and ethyl acetate (5 ml) were added and the mixture was heated to 80° C. with stirring for 1 hr. The solid was filtered at 80° C. and the cake washed with 1,4-dioxane (10 ml) at 80° C. To the wet cake ethyl acetate (100 ml) was added and the mixture was refluxed with stirring for 1 hr, then the mixture was cooled to 25° C. to 30° C., filtered to obtain a solid, and the solid was washed with ethyl acetate (20 ml) and dried under vacuum to afford the titled compound (7 g). Yield: 77.7% Purity: 94.88%

### Example 6: Preparation of 4-amino-2-(2,6-dioxopiperidin-3-yl) isoindoline-1,3-dione

A round bottomed flask was charged with 1,4-dioxane (600 ml) and 2-(2,6-dioxopiperidin-3-yl)-4-nitroisoindoline-1,3-dione (10 g). The reaction mass was stirred at room temperature (25° C. to 30° C.) for 15 minutes and the temperature raised up to 80° C. and the reaction mass stirred at this temperature to get a clear solution. To the clear solution activated charcoal (5 g) was added at 80° C. and the mass was cooled to 25° C. to 30° C. The reaction mass was stirred at this temperature for 20 minutes and filtered. To the obtained solution 1,4-dioxane (300 ml), water (180 ml) and acetic acid (1.2 ml) were added followed by addition of Pd/C (1.0 g 10%). The reaction mixture was stirred at 20° C. to 30° C. under 70 psi hydrogen pressure for 12 hrs. The reaction mass was filtered through a celite bed and the solvent was distilled off from the reaction mass under vacuum at 50° C. to get the solid. To the solid obtained 1,4-dioxane (50 ml) and ethyl acetate (5 ml) were added and the mixture was heated to 80° C. with stirring for 1 hr. The solid was filtered at 80° C. and the cake washed with 1,4-dioxane (10 ml) at 80° C. To the wet cake ethyl acetate (100 ml) was added and the mixture was refluxed with stirring for 1 hr, then the mixture was cooled to 25° C. to 30° C., filtered, and the solid washed with ethyl acetate (20 ml) and dried under vacuum to afford the titled compound (7 g). Yield: 77.7% Purity: 99%

### Example 7: Preparation of 4-amino-2-(2,6-dioxopiperidin-3-yl) isoindoline-1,3-dione

A round bottomed flask was charged with 1,4-dioxane (600 ml) and 2-(2,6-dioxopiperidin-3-yl)-4-nitroisoindoline-1,3-dione (10 g). The reaction mass was stirred at room temperature (25° C. to 30° C.) for 15 minutes and the temperature raised up to 80° C. and the reaction mass stirred at this temperature to get a clear solution. To the clear solution activated charcoal (5 g) was added at 80° C. and the mass was cooled to 25° C. to 30° C. The reaction mass was stirred at this temperature for 20 minutes and filtered. To the obtained solution 1,4-dioxane (300 ml) and water (180 ml) were added followed by addition of Pd/C (1.0 g 10%). The reaction mixture was stirred at 20° C. to 30° C. under 70 psi hydrogen pressure for 12 hr. The reaction mass was filtered through a celite bed and the solvent was distilled off from the reaction mass under vacuum at 50° C. to get the solid. To the solid obtained 1,4-dioxane (50 ml) and ethyl acetate (5 ml) were added and the mixture was heated to 80° C. with stirring for 1 hr. The solid was filtered at 80° C. and the cake washed with 1,4-dioxane (10 ml) at 80° C. To the wet cake ethyl acetate (100 ml) was added and the mixture was refluxed with stirring for 1 hr, then the mixture was cooled to 25° C. to 30° C., filtered, the solid was washed with ethyl acetate (20 ml) and dried under vacuum to afford the titled compound (7 g). Yield: 77.7% Purity: 99%

### Example 8: Preparation of 4-amino-2-(2,6-dioxopiperidin-3-yl) isoindoline-1,3-dione

Acetic acid (1 L) was added to 2-(2,6-dioxopiperidin-3-yl)-4-nitroisoindoline-1,3-dione (10 g) in a round bottomed flask. The mixture was stirred at 25° C. to 30° C. for 15 minutes, the temperature raised up to 90° C. and the reaction mass stirred to get a clear solution. To the obtained solution Pd/C (1.0 g 10%) was added and the reaction mixture was stirred at 90° C. under 70 psi hydrogen for 2 hr. The reaction mass was then filtered through a celite bed and the solvent was distilled off from the reaction mass under vacuum at 50° C. to get the solid. To the obtained solid water (50 ml) was added at 30° C. The reaction mass was stirred for 1 hr, filtered to obtain the solid and the solid washed with water (10 ml). To the wet cake ethyl acetate (100 ml) was added and the mixture was refluxed with stirring for 1 hour. Then the mass was cooled to 25° C. to 30° C., filtered to obtain the solid. The solid was washed with ethyl acetate (20 ml) and dried under vacuum to afford the titled compound (7 g). Yield: 77.7% Purity: 99%

### Example 9: Preparation of 4-amino-2-(2,6-dioxopiperidin-3-yl) isoindoline-1,3-dione

A round bottomed flask was charged with tetra methyl urea (200 ml) and 2-(2,6-dioxopiperidin-3-yl)-4-nitroisoindoline-1,3-dione (10 g). The reaction mass was stirred at 25° C. to 30° C. for 15 minutes. The reaction mass was heated up to 80° C. to 90° C. and stirred at this temperature to get a clear solution. To this clear solution activated charcoal (5 g) was added at 80° C. to 90° C. and the mass was cooled to 25° C. to 30° C. The reaction mass was then stirred at this temperature for 20 minutes and filtered. To the obtained solution Pd/C (1.0 g 10%) was added and the reaction mixture was stirred at 20° C. to 30° C. under 70 psi hydrogen pressure for 6 hr. After completion of reaction, the reaction mass was filtered through a celite bed and the bed was washed with tetra methyl urea (20 ml). To this reaction mass water (300 ml) was added, the mass stirred for 3 hr at 25° C. to 30° C. The precipitated solid was filtered and washed with water (2×20 ml). The wet cake was slurried with water (200 ml), the slurry filtered and the wet cake dried to afford the titled compound (7 g). Yield: 77% Purity: 99% (TLC)

### Purification of 4-amino-2-(2,6-dioxopiperidin-3-yl) isoindoline-1,3-dione (pomalidomide)

### Example 10: Purification of 4-amino-2-(2,6-dioxopiperidin-3-yl) isoindoline-1,3-dione

Pomalidomide crude (10 g) as obtained in Example 2 was suspended in 1,4-dioxane (100 ml) and ethyl acetate (5 ml). The suspension was heated to 85° C. with stirring for 5 hrs. The reaction mass was filtered, the wet cake washed with 1,4-dioxane at 85° C. The wet cake was then charged in 1,4-dioxane (100 ml) and ethyl acetate (10 ml) then heated to 85° C. with stirring for 5 hours. The reaction mass was filtered, the wet cake washed with ethyl acetate (100 ml) at 85° C. and the wet cake was dried under vacuum at 50° C. until LOD was less than 0.5%. Purity: 99.6%

### Example 11

4-amino-2-(2,6-dioxopiperidin-3-yl) isoindoline-1,3-dione (10 g), as obtained in Example 2, was added to tetra methyl urea (200 ml) and the mixture was stirred at 100° C. to get a clear solution. The solution was cooled to 50° C. and activated charcoal (1.0 g) was added to it. The mixture was stirred for 1 hr, then filtered through a celite bed and washed with tetra methyl urea (2 ml) at 50° C. The solution was heated at 80° C. and water (303 ml) was slowly added to the solution at 80° C. with stirring at 100 to 150 revolutions per minute. After completion of water addition, slowly the reaction mass was cooled to 35° C. to 40° C. The mixture was stirred at 35° C. to 40° C. for 4 hr and the solid filtered and washed with water (20 ml) at 40° C. The wet cake was charged in preheated water (100 ml×3) at 50° C., stirred for 1 hr at 50° C., the solid filtered at 50° C., washed with water (20 ml) and the wet cake dried in an air tray dryer until water content was less than 0.5%. The dried solid was charged in acetone (100 ml) and the mixture refluxed with stirring for 1 hr, then cooled to room temperature, the solid filtered, washed with acetone (20 ml) and dried under vacuum at 50° C. until LOD was less than 0.5% to afford pure pomalidomide (8.5 g). Pomalidomide obtained was a crystalline form having powder x-ray diffraction spectrum as shown in FIG. 2. Purity: 99.88%

### Example 12

Pomalidomide crude (10 g) was suspended in tetra methyl urea (50 ml) and the suspension was heated to 100° C. with stirring for 3 hr to get a clear solution. The solution was cooled to 30° C., stirred for 2 hr at 25° C. to 30° C., filtered and washed with tetra methyl urea (5 ml). The wet cake was washed with water (50 ml) and filtered. The obtained wet cake was dried to get pure pomalidomide (7.2 g). Purity: 99.9%

### Example 13

Pomalidomide crude (10 g) was suspended in tetra methyl urea (200 ml) and the suspension was heated to 100° C. with stirring to get clear solution. This solution was subjected to carbon treatment at 80° C. To the reaction mass methanol (600 ml) was added at 40° C. The mixture was stirred for 3 hr at 35° C. to 40° C. The separated solid was filtered and then dried to obtain pure pomalidomide (7.2 g). Purity: 99.9%

### Example 14

Pomalidomide crude (10 g) was suspended in tetra methyl urea (200 ml) and the suspension was heated to 100° C. with stirring to get a clear solution. This solution was subjected to carbon treatment at 80° C. To the reaction mass acetone (200 ml) was added at 40° C. and the reaction mass was stirred for 30 minutes at 35° C. to 40° C. To the reaction mass water (200 ml) was added at 35° C. to 40° C., the mixture was stirred at the same temperature for 3 hr. The separated solid was filtered, washed with a mixture of acetone (100 ml) and water (100 ml) and dried to afford pure pomalidomide (7.4 g). Purity: 99.9%

### Example 15

Pomalidomide crude (10 g) was suspended in tetra methyl urea (200 ml) and the suspension was heated to 100° C. with stirring to get a clear solution. This solution was subjected to carbon treatment at 80° C. To the reaction mass cyclohexane (600 ml) was added at 40° C., the reaction mass was stirred for 3 hr at 35° C. to 40° C. The separated solid was filtered and then dried to afford pure pomalidomide (7.2 g). Purity: 99.9%

### Example 16

Pomalidomide crude (10 g) was suspended in tetra methyl urea (200 ml) and the suspension was heated to 100° C. with stirring to get a clear solution. This solution was subjected to carbon treatment at 80° C. To the reaction mass diisopropyl ether (600 ml) was added at 40° C. The reaction mass was stirred for 3 hr at 35° C. to 40° C. The separated solid was filtered and then dried to afford pure pomalidomide (7.2 g). Purity: 99.9%

### Example 17

Pomalidomide crude (10 g) was suspended in tetra methyl urea (200 ml) and the suspension was heated to 100° C. with stirring to get a clear solution. This solution was subjected to carbon treatment at 80° C. To the reaction mass ethyl acetate (600 ml) was added at 40° C. The reaction mass was stirred for 3 hr at 35° C. to 40° C. The separated solid was filtered and then dried to afford pure pomalidomide (7.2 g). Purity: 99.9%

### Example 18

Pomalidomide crude (10 g) was suspended in tetra methyl urea (200 ml) and the suspension was heated to 100° C. with stirring to get a clear solution. This solution was subjected to carbon treatment at 80° C. To the reaction mass toluene (600 ml) was added at 40° C. The reaction mass was stirred for 3 hr at 35° C. to 40° C. The separated solid was filtered and then dried to afford pure pomalidomide (7.2 g). Purity: 99.9%

### Example 19

Pomalidomide crude (10 g) was suspended in tetra methyl urea (200 ml) and the suspension was heated to 100° C. with stirring to get a clear solution. This solution was subjected to carbon treatment at 80° C. To the reaction mass methyl tertiary butyl ether (800 ml) was added at 40° C. The reaction mass was stirred for 3 hr at 35° C. to 40° C. The separated solid was filtered and then dried to afford pure pomalidomide (7.2 g). Purity: 99.9%

### Example 20

Pomalidomide crude (10 g) was suspended in tetra methyl urea (200 ml) and the suspension was heated to 100° C. to get a clear solution. This solution was subjected to carbon treatment at 80° C. To the reaction mass acetic acid (600 ml) was added at 25° C. The reaction mass was stirred for 3 hr at 20° C. to 25° C. The separated solid was filtered and then dried to afford pure pomalidomide (7.2 g). Purity: 99.9%

Although the methods and compounds of the present disclosure have been described with reference to exemplary embodiments thereof, the present disclosure is not limited thereby. Indeed, the exemplary embodiments are provided for illustrative and non-limitative purposes. Changes, modifications, enhancements and/or refinements to the disclosed methods may be made without departing from the spirit or scope of the present disclosure. Accordingly, such changes, modifications, enhancements and/or refinements are encompassed within the scope of the present invention.

What is claimed is:

1. A process for making pomalidomide comprising reducing 2-(2,6-dioxopiperidin-3-yl)-4-nitroisoindoline-1,3-dione using a catalyst and at least one solvent selected from 1,4-dioxane, water, acetic acid, tetramethylurea (TMU), N,N′-dimethyl ethylene urea (DMEU), N,N-dimethyl propylene urea (DMPU) or mixtures thereof to obtain 4-amino-2-(2,6-dioxopiperidin-3-yl) isoindoline-1,3-dione (pomalidomide), and optionally purifying the pomalidomide.

2. The process of claim 1 further comprising reacting 3-nitrophthalic anhydride with α-amino glutarimide hydrochloride to obtain the 2-(2,6-dioxopiperidin-3-yl)-4-nitroisoindoline-1,3-dione.

3. The process according to claim 1 further comprising, prior to the reducing step, subjecting the 2-(2,6-dioxopiperidin-3-yl)-4-nitroisoindoline-1,3-dione to a purification process comprising heating a mixture of 2-(2,6-dioxopiperidin-3-yl)-4-nitroisoindoline-1,3-dione and 1,4-dioxane to obtain a solution, treating the obtained solution with carbon, removing the carbon from the solution to obtain a purified 2-(2,6-dioxopiperidin-3-yl)-4-nitroisoindoline-1,3-dione solution and using the purified 2-(2,6-dioxopiperidin-3-yl)-4-nitroisoindoline-1,3-dione solution for the reducing step.

4. The process of claim 1 wherein the catalyst is palladium on carbon (Pd/C) or Raney nickel and the at least one solvent is selected from the group consisting of 1,4-dioxane, water, acetic acid and mixtures thereof.

5. The process of claim 2 wherein the reaction of 3-nitrophthalic anhydride with α-amino glutarimide hydrochloride is carried out in the presence of sodium acetate and acetic acid.

6. The process according to claim 4 wherein the catalyst is Pd/C and the solvent is 1,4-dioxane.

7. The process according to claim 4 wherein the catalyst is Pd/C or Raney nickel and the solvent is a mixture of 1,4-dioxane and water.

8. The process according to claim 4 wherein the catalyst is Pd/C and the solvent is a mixture of 1,4-dioxane, water and acetic acid.

9. The process according to claim 4 wherein the catalyst is Pd/C and the solvent is acetic acid.

10. The process according to claim 1 wherein the solvent is TMU.

11. The process according to claim 1 wherein the reaction time for reduction using a palladium catalyst is in the range of 1 to 12 hr.

12. The process according to claim 1 wherein the reaction time for reduction using a Raney nickel catalyst is in the range of 60 to 90 hr.

13. The process according to claim 1 further comprising subjecting the pomalidomide to a purification process comprising suspending pomalidomide in one or more of 1,4-dioxane, water, ethyl acetate or a mixture thereof, optionally heating the suspension, and recovering purified pomalidomide from the suspension.

14. The process according to claim 13 comprising suspending the pomalidomide in a mixture of 1,4-dioxane and ethyl acetate, heating the suspension, filtering the suspension to obtain a wet pomalidomide material; combining the wet pomalidomide material with ethyl acetate under reflux conditions, stirring the pomalidomide material combined with the ethyl acetate, cooling the stirred material to room temperature, filtering and subsequently washing the cooled material to obtain purified pomalidomide.

15. The process of claim 1 further comprising subjecting the pomalidomide to a purification process comprising the steps of a) suspending pomalidomide in TMU to obtain a suspension, b) heating the suspension to obtain a clear solution, c) treating the clear solution obtained in step b) with carbon, then removing the carbon prior to step d), d) adding an anti-solvent to the solution obtained in step c), and e) recovering purified pomalidomide.

16. The process of claim 15 wherein the ratio of pomalidomide to TMU is about 20 volumes of TMU to one gram of pomalidomide.

17. The process of claim 15 wherein the anti-solvent used in step d) is water, methanol, acetone, cyclohexane, diisopropyl ether, ethyl acetate, toluene, methyl tertiary butyl ether, acetic acid or a mixture thereof.

18. The process of claim 1 further comprising subjecting the pomalidomide to a purification process comprising the steps of a) suspending pomalidomide in TMU, b) heating the suspension, c) subsequently cooling the suspension, d) stirring the cooled suspension, e) subjecting the suspension of step d) to filtration to obtain a solid product, f) leaching the solid product with water, and g) recovering purified pomalidomide.

19. The process of claim 18 wherein the ratio of pomalidomide to TMU is about 5 volumes of TMU to one gram of pomalidomide.

20. Pomalidomide made according to claim 1 having a purity of greater than 99% as measured by HPLC with no individual impurity present in an amount greater than 0.1% and total impurities comprising not more than 0.5%.

21. Pomalidomide made according to claim 1 having a purity of 95% or greater as measured by high pressure liquid chromatography.

22. Pomalidomide made according to claim 1 having a purity of greater than 98% as measured by high pressure liquid chromatography.
